(19) United States
(12) Patent Application Publication
(10) Pub. No.: US 2010/0099725 A1
(43) Pub. Date: Apr. 22, 2010

(54) PEROXISOME PROLIFERATOR ACTIVATED RECEPTOR MODULATORS

(76) Inventors: Lance Allen Pfeifer, Carmel, IN (US); Tianwei Ma, Carmel, IN (US); Nathan Bryan Mantlo, Brownsburg, IN (US); Laura Frey Michael, Indianapolis, IN (US); Fese Mambo Mokube, Fishers, IN (US); Chahrzad Montrose-Rafizadeh, Indianapolis, IN (US)

Correspondence Address: ELI LILLY & COMPANY, PATENT DIVISION, P.O. BOX 6288, INDIANAPOLIS, IN, 46206-6288, US

(21) Appl. No.: 12/522,723

(22) PCT Filed: Feb. 12, 2008
(86) PCT No.: PCT/US08/53653

§ 371 Date: Jul. 10, 2009

Related U.S. Application Data
(60) Provisional application No. 60/891,261, filed on Feb. 23, 2007.

(51) Int. Cl. A61K 31/4196; C07D 249/12; A61P 9/10

(52) U.S. Cl. 514/384; 548/263.2

(57) ABSTRACT

The present invention is directed to compounds of the structural Formula: wherein: R¹ is H or —C₁-C₃ alkyl; R² is selected from the group consisting of —H, —C₁-C₄ alkyl, —C₁-C₃ alkyl-CF₃, phenyl, and pyridinyl; and R³ is selected from the group consisting of —H, —C₁-C₄ alkyl, —C₁-C₃ alkyl-O—CH₃, —CH₂-cyclopropyl, CH₂—C═CH₂, —CH₂CH₂-(2-F-phenyl), and phenyl substituted with from 1 to 2 fluorines; provided that when R¹ and R² are each H, then R³ is selected from the group consisting of —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines; or stereoisomers and pharmaceutically acceptable salts thereof.

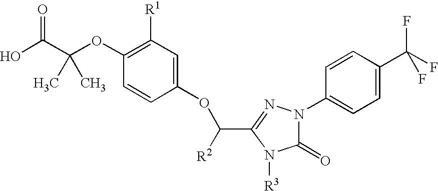

### CROSS-REFERENCE

This application claims the benefit of U.S. Provisional Application No. 60/891,261, filed Feb. 23, 2007.

### BACKGROUND OF THE INVENTION

Peroxisome Proliferator Activated Receptors (PPARs) are members of the nuclear hormone receptor super family, which are ligand-activated transcription factors regulating gene expression. Various subtypes of PPARs have been discovered and are reported to be targets for the development of new therapeutic agents. The PPAR receptors include PPARα, PPARγ and PPARδ. The PPARα and PPARβ receptors have been implicated in diabetes mellitus, cardiovascular disease, obesity, and inflammation. Compounds modulating both the PPARα and PPARδ receptors are believed to be especially useful for cardiovascular disease; for example, hyperlipidemia, hypertriglyceridemia, and atherosclerosis. PPARα is the target of currently marketed hyperlidemic fibrate drugs which reportedly produce a substantial reduction in plasma triglycerides and moderate reduction in low density lipoprotein (LDL) cholesterol. Compounds disclosed in WO200338553 are reported to be agonists of the PPARα receptor.

PPARδ agonism is a therapeutic target for hypertriglyceridemia and insulin resistance. PPARδ agonists have been disclosed as a potential treatment for use in regulating many of the parameters associated with metabolic syndrome and atherosclerosis. It has been reported that in obese, non-diabetic rhesus monkeys, a PPARδ agonist reduced circulating triglycerides and LDL cholesterol, decreased basal insulin levels and increased HDL cholesterol. The increase in HDL cholesterol correlated with an increase in the number of HDL particles, there was an increase in the serum levels of HDL-associated apolipoproteins apoA-I, apoA-II, and apoC-III, and fasting insulin levels decreased. Treatments targeting PPARδ agonist activity are desired to provide additional treatment options for both cardiovascular disease and insulin resistance. Current treatments for cardiovascular disease and conditions associated with metabolic syndrome are often co-administered with other pharmaceutical agents.

Compounds that modulate both PPARα and PPARδ, while offering an acceptable safety profile for co-administration with other treatments, would be particularly desirable. PPAR agonists having low PXR modulation may minimize undesired drug-drug interactions. Drugs given concomitantly with other drugs or even in combination with plant extracts such as St. John's wort or grapefruit juice have the potential to cause inefficacy of drug treatment or adverse drug reactions. Therefore, knowledge of the enzymes that metabolize certain compounds combined with knowledge of its inducers and inhibitors is a common feature of package inserts or drug information sheets to anticipate and prevent these adverse effects. For example, it has been reported that problems associated with the antidiabetic drug troglitazone (Rezulin™) could partially be explained by the discovery that it activated the Pregnane X Receptor (PXR) in addition to its effect on PPAR. Subsequently, a troglitazone related compound, rosiglitazone, was negatively tested for PXR activation. Rosiglitazone is currently marketed in the U.S. as the drug Avandia™, while troglitazone was removed from the U.S. market due to safety concerns. Thus, the skilled artisan is faced with the problem of providing new PPARδ and PPARα agonists having an acceptable safety profile, and offering low drug-drug interaction with other pharmaceutical agents, as suggested by low PXR activity.

This invention provides potent dual agonsists of PPARδ and PPARα. This invention also provides PPARδ and PPARα dual agonists that demonstrate low PXR modulation using the PPAR and PXR assay methods discussed herein. Compounds of this invention may provide the desired treatments for cardiovascular disease and insulin resistance, as shown by the PPAR receptor activity, while minimizing the incidence of undesired drug-drug interactions, as demonstrated by the low PXR activation.

### BRIEF SUMMARY OF THE INVENTION

The present invention is directed to compounds represented by the following structural Formula I:

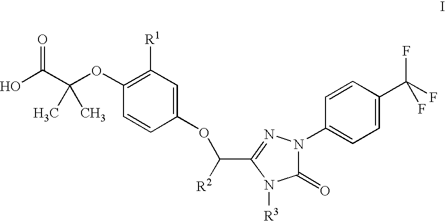

wherein:

R¹ is —H or —C₁-C₃ alkyl;

R² is selected from the group consisting of —H, —C₁-C₄ alkyl, —C₁-C₃ alkyl-CF₃, phenyl, and pyridinyl; and

R³ is selected from the group consisting of —H, —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines;

provided that when R¹ and R² are each H, then R³ is selected from the group consisting of —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines; or

stereoisomers and pharmaceutically acceptable salts thereof.

In another embodiment, the invention provides a compound structurally represented by formula I, wherein:

R¹ is —H or —CH₃;

R² is selected from the group consisting of —H, —C₁-C₄ alkyl, —C₁-C₃ alkyl-CF₃, phenyl, and pyridinyl; and

R³ is selected from the group consisting of —H, —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with 1 or 2 fluorines;

provided that when R¹ and R² are each H, then R³ is selected from the group consisting of —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines; or

stereoisomers and pharmaceutically acceptable salts thereof.

In another embodiment, the invention provides a compound structurally represented by formula I, wherein:

R¹ is —H or —CH₃;

R² is selected from the group consisting of —H, —CH₃, —CH₂CH₃, —CH₂CH₂CH₃, —CH₂CH₂CH₂CH₃, —CH(CH3)₂, —CH₂CH₂CF₃, —CH₂CH₂CH₂CF₃, phenyl, and 3-pyridinyl; and

R³ is selected from the group consisting of —H, —CH₃, —CH₂CH₃, —CH₂CH₂CH₃, —CH(CH₃)₂, —CH₂—C═CH₂, —CH₂CH₂—O—CH₃, —CH₂CH(CH₃)₂, —C(CH₃)₃, —CH₂-cyclopropyl, 2,6-diF-phenyl, 2-F-phenyl, and —CH₂CH₂-(2-F-phenyl);

provided that when R¹ and R² are each H, then R³ is selected from the group consisting of —CH₂CH₃, —CH(CH₃)₂, 2-F-phenyl, and 2,6-diF-phenyl; or

stereoisomers and pharmaceutically acceptable salts thereof.

In another embodiment, the invention provides a compound structurally represented by formula I, wherein:

R¹ is —H or —CH₃;

R² is selected from the group consisting of —H, —C₁-C₄ alkyl, and —C₁-C₃ alkyl-CF₃; and

R³ is selected from the group consisting of —C₁-C₄ alkyl, —CH₂-cyclopropyl, —CH₂—C═CH₂, and phenyl substituted with 1 or 2 fluorines;

provided that when R¹ and R² are each H, then R³ is selected from the group consisting of —C₁-C₄ alkyl, —CH₂-cyclopropyl, —CH₂—C═CH₂, and phenyl substituted with from 1 to 2 fluorines; or

stereoisomers and pharmaceutically acceptable salts thereof.

In another embodiment, the invention provides a compound structurally represented by formula I, wherein:

R¹ is —H or —CH₃;

R² is selected from the group consisting of —H, —CH₃, —CH₂CH₃, —CH₂CH₂CH₃, —CH₂CH₂CH₂CH₃, and —CH₂CH₂CH₂CF₃; and

R³ is selected from the group consisting of —CH₂CH₂CH₃, —CH(CH₃)₂, —CH₂—C═CH₂, —CH₂-cyclopropyl, 2,6-diF-phenyl, or 2-F-phenyl; or

stereoisomers and pharmaceutically acceptable salts thereof.

In another embodiment, the invention provides a compound structurally represented by formula I, wherein:

R¹ is —H or —CH₃;

R² is —CH₂CH₂CH₃ or —CH₂CH₂CH₂CH₃; and

R³ is -2-F-phenyl or 2,6-diF-phenyl; or

stereoisomers and pharmaceutically acceptable salts thereof.

In another embodiment, the invention provides a compound structurally represented by formula I;

wherein:

R¹ is —H;

R² is selected from the group consisting of —H, —CH₃, —CH₂CH₃, —CH₂CH₂CH₃, —CH₂CH₂CH₂CH₃, —CH₂CH₂CF₃, —CH₂CH₂CH₂CF₃, phenyl, and 3-pyridinyl; and

R³ is selected from the group consisting of —CH₃, —CH₂CH₃, —CH(CH₃)₂, 2-F-phenyl, 2,6-diF-phenyl, or —CH₂CH₂-(2-F-phenyl);

provided that when R¹ and R² are each H, then R³ is selected from the group consisting of 2-F-phenyl, —CH₂CH₃, 2,6-diF-phenyl, and —CH(CH₃)₂; or

stereoisomers and pharmaceutically acceptable salts thereof.

In another embodiment, the invention provides a compound structurally represented by formula I;

wherein:

R¹ is —CH₃;

R² is selected from the group consisting of —H, —CH₃, —CH₂CH₂CH₃, —CH₂CH₂CH₂CH₃, —CH₂CH₂CF₃, —CH₂CH₂CH₂CF₃, phenyl, and 3-pyridinyl; and

R³ is selected from the group consisting of —CH₃, —CH₂CH₃, —CH₂CH₂CH₃, —CH₂—C═CH₂, —CH(CH₃)₂, —CH₂CH(CH₃)₂, —C(CH₃)₃, —CH₂CH₂—O—CH₃, —CH₂-cyclopropyl, 2,6-diF-phenyl, 2-F-phenyl, and —CH₂CH₂-(2-F-phenyl); or

stereoisomers and pharmaceutically acceptable salts thereof.

In another embodiment, the present invention also relates to pharmaceutical formulations comprising at least one compound of the present invention, or a pharmaceutically acceptable salt or stereioisomer thereof, and a pharmaceutically acceptable carrier.

In another embodiment, the present invention relates to a method of selectively modulating a PPARδ receptor and PPARα receptor, as compared to other PPAR receptor subtypes, yet having little stimulatory effect on the Pregnane X Receptor, by contacting the respective receptors with at least one compound represented by Structural Formula I or a pharmaceutically acceptable salt or stereioisomer thereof.

In another embodiment, the present invention provides an intermediate of Formula IV:

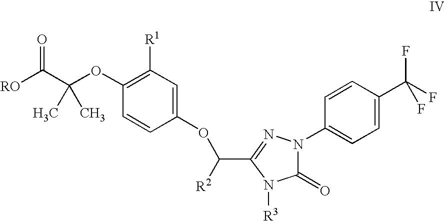

wherein:

R is —C₁-C₃ alkyl;

R¹ is —H or —C₁-C₃ alkyl;

R² is selected from the group consisting of —H, —C₁-C₄ alkyl, —C₁-C₃ alkyl-CF₃, phenyl, and pyridinyl; and

R³ is selected from the group consisting of —H, —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines;

provided that when R¹ and R² are each H, then R³ is selected from the group consisting of —C₁-C₄ alkyl, —C₁-C₃ alkyl-O—CH₃, —CH₂-cyclopropyl, —CH₂—C═CH₂, or phenyl substituted with from 1 to 2 fluorines; or

stereoisomers and pharmaceutically acceptable salts thereof.

Choice of the appropriate chiral column, eluent and conditions necessary to effect separation of the enantiomeric pair is well within the knowledge of one of ordinary skill in the art. In addition, the specific stereoisomers and enantiomers of compounds of formula I can be prepared by one of ordinary skill in the art utilizing well known techniques and processes, such as those disclosed by J. Jacques, et al., “Enantiomers, Racemates, and Resolutions”, John Wiley and Sons, Inc., 1981, and E. L. Eliel and S. H. Wilen,” Stereochemistry of Organic Compounds”, (Wiley-Interscience 1994), and European Patent Application No. EP-A-838448, published Apr. 29, 1998. Examples of resolutions include recrystallization techniques or chiral chromatography.

Compounds of the present invention have a chiral center and may exist in a variety of stereoisomeric configurations. As a consequence of this chiral center, the compounds of the present invention occur as racemates, mixtures of enantiomers and as individual enantiomers. All such racemates and enantiomers are within the scope of the present invention. The examples herein are particularly preferred compounds of the invention. “Pharmaceutically-acceptable salt” refers to salts of the compounds of the invention considered to be acceptable for clinical and/or veterinary use. These salts may be prepared by methods known to the skilled artisan. Pharmaceutically acceptable salts and common methodology for preparing them are well known in the art. See, e.g., P. Stahl, et al., HANDBOOK OF PHARMACEUTICAL SALTS: PROPERTIES, SELECTION AND USE, (VCHA/Wiley-VCH, 2002); S. M. Berge, et al.,

“Pharmaceutical Salts,” Journal of Pharmaceutical Sciences, Vol. 66, No. 1, January 1977. The compounds of the present invention are preferably prepared as pharmaceutical compositions administered by a variety of routes. The term “pharmaceutically acceptable” means that the carrier, diluent, excipients and salt are pharmaceutically compatible with the other ingredients of the composition. Most preferably, such formulations are for oral administration. Such pharmaceutical formulations and processes for preparing same are well known in the art. See, e.g., REMINGTON: THE SCIENCE AND PRACTICE OF PHARMACY (A. Gennaro, et al., eds., 19^{th} ed., Mack Publishing Co., 1995).

### DETAILED DESCRIPTION OF THE INVENTION

Definitions: THF is tetrahydrofuran, EtOAc is ethyl acetate, Et₂O is diethyl ether, DEAD is diethyl azodicarboxylate, PPh₃ is triphenylphosphine, ADDP is 1,1-(azodicarbonyl)-dipiperidine, Bu₃P is tri-n-butylphosphine, DIPEA is N,N-diisopropylethylamine, BBr₃ is boron tribromide, TMSOTf is trimethylsilyl trifluoromethanesulfonate, Pd(OH)₂/C is palladium hydroxide on carbon, and Bn is benzyl.

The term “alkyl” unless otherwise indicated, refers to those alkyl groups of a designated number of carbon atoms of either a straight or branched saturated configuration. C₁-C₃ alkyl refers to methyl, ethyl, n-propyl and isopropyl. C₁-C₄ alkyl refers to methyl, ethyl, n-propyl, isopropyl, n-butyl, isobutyl, sec-butyl, and tert-butyl. The preparations and examples are named using AutoNom 2000 in MDL ISIS/Draw version 2.5 SP1 from MDL Information Systems, Inc.

The method employed in the synthesis of the Examples of the present invention is illustrated in Scheme 1. Generally, an alcohol intermediate of Formula II reacts with a phenol intermediate of Formula III under Mitsunobu conditions (DEAD/PPh₃, ADDP/Bu₃P etc.) to form the ester of Formula IV. For the preparation of the phenol compounds of Formula II, see WO2001016120 and WO2004063166. Hydrolysis in the presence of aqueous NaOH or LiOH gives compounds of Formula I.

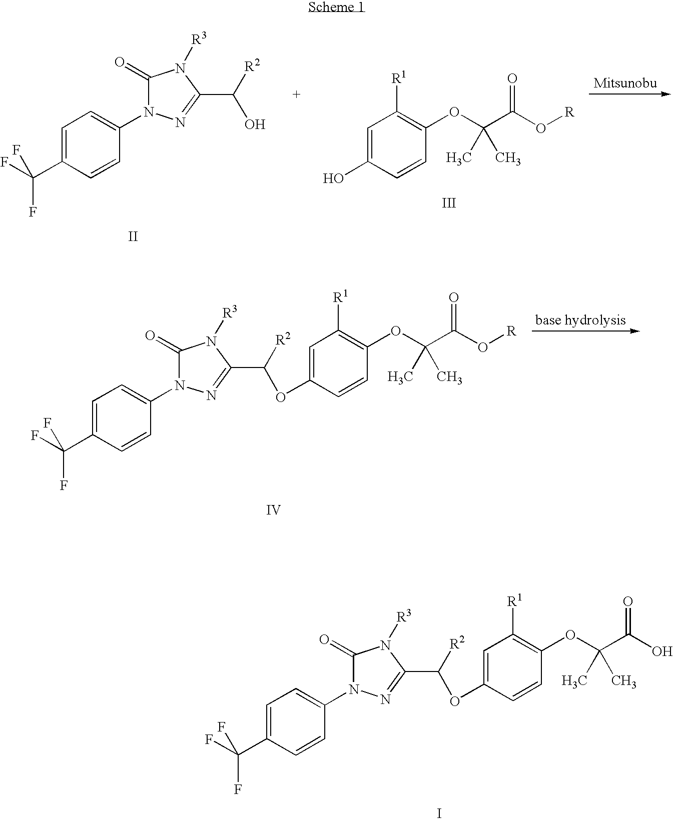

Under certain circumstances, the synthetic sequence can be altered as shown in Scheme 2, where a protective group (PG), such as allyl, at the N-4 position of triazolone is utilized. Deprotection followed by alkylation with R³X in the presence of base (K₂CO₃, NaH, DIPEA, etc.) gives the penultimate ester that is hydrolysed in the presence of aqueous base (NaOH or LiOH) to give the acid product.

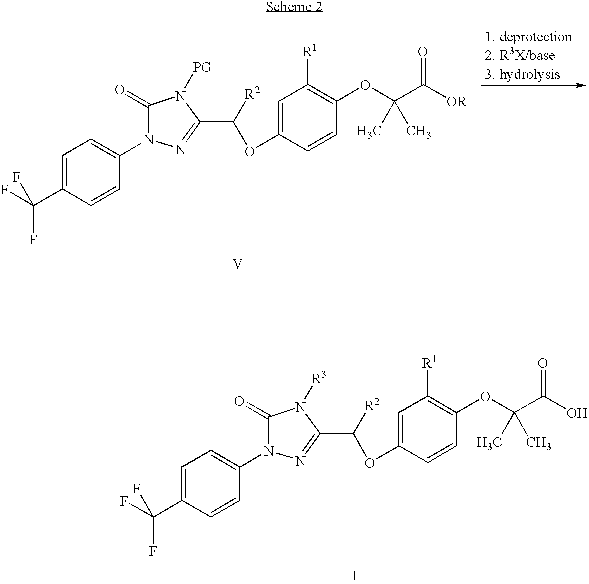

The alcohol intermediate Formula II is prepared as shown in Scheme 3 (via thioimidate) and is referred to as the Alcohol Intermediate Route A). The α-benzyloxyamide compound 2, obtained from its acid or acyl chloride precursor, is converted to the thioamide compound 3. Alkylation with methyl triflate or methyl iodide results in the thioimidate derivative compound 4, which is further treated with a phenyl hydrazine derivative followed by carbodiimidazle to give compound 6. Debenzylation of compound 6 with BBr₃ or hydrogenolysis gives the primary alcohol intermediate compound of Formula IIa.

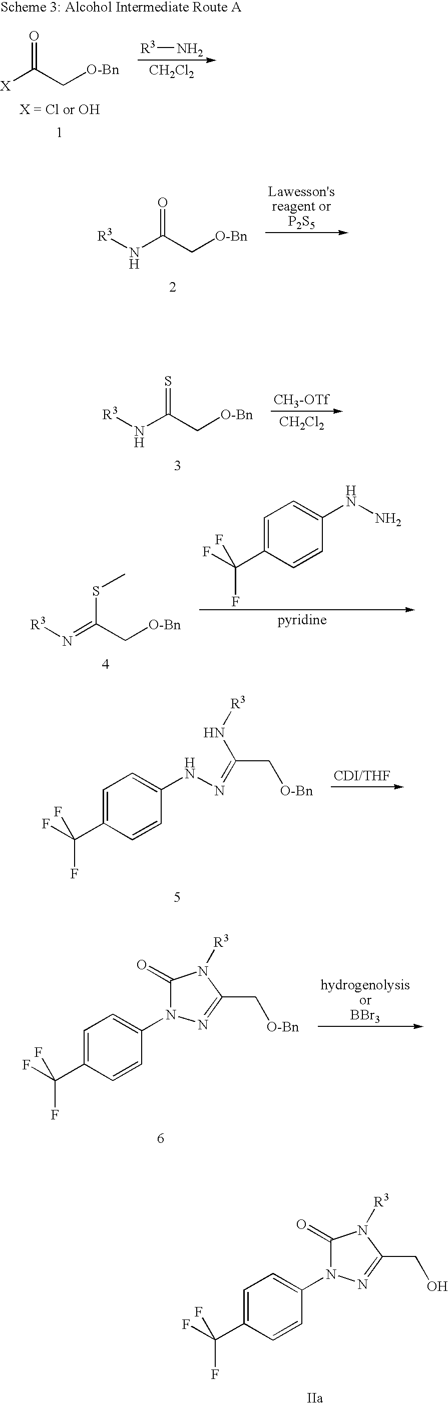

Alternatively, intermediate 6 can be prepared using the method shown in Scheme 4 (via a semicarbazide) and is referred to as Alcohol Intermediate Route B. For example, compound 1a is converted to its acylhydrazide derivative compound 7, which is treated with an isocyanate followed by TMOSTf to give the triazolone derivative compound 9. Coupling under the Buchwald conditions with an aryl halide gives compound 6. Then, debenzylation of compound 6 with BBr₃ or hydrogenolysis gives the primary alcohol intermediate compound of Formula IIa.

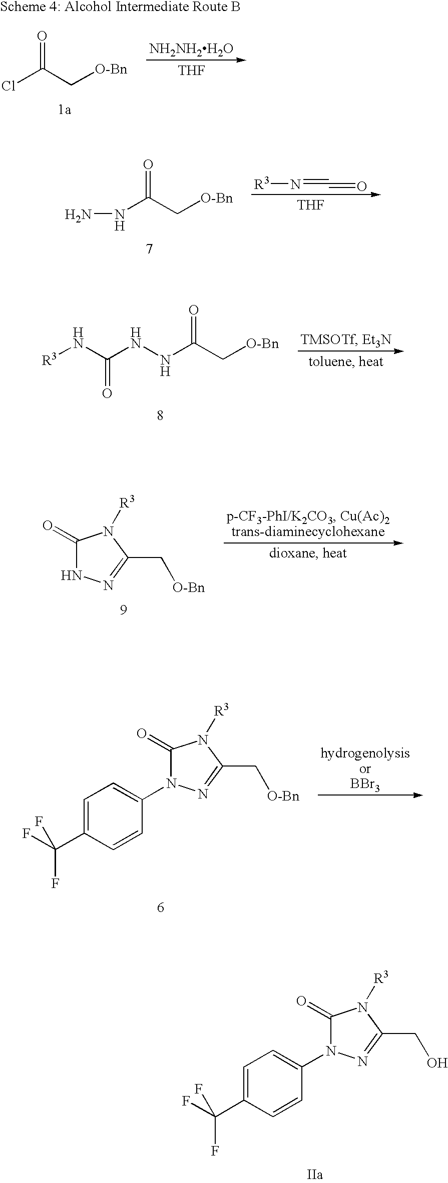

In Scheme 5, compounds of Formula IIa can be optionally oxidized to the aldehyde compound 8 which is then converted to the secondary alcohol compound 9 via addition of a Grignard reagent, R²MgX.

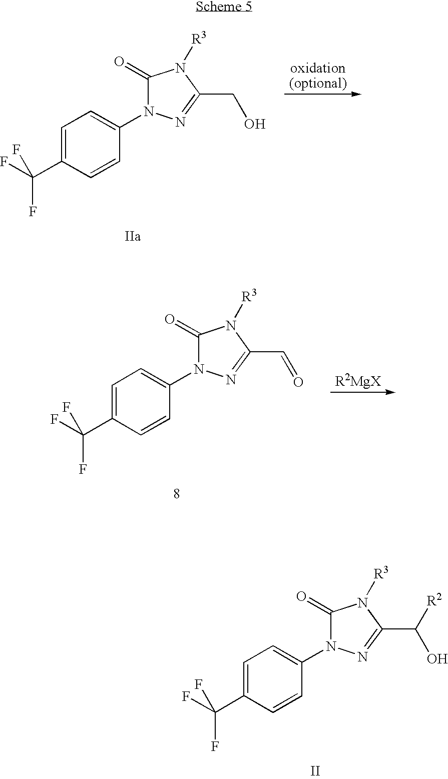

When R² is a substituent other than hydrogen, then there is a chiral center at the carbon where R² is attached as shown below.

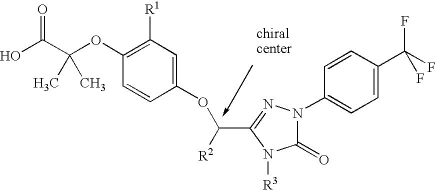

For compounds where R² is other than hydrogen, the ester protected penultimate intermediate (for example, Formula IV) is racemic. At this point, the racemic mixture is separated by chiral chromatography into the two isomers, Isomer 1 and Isomer 2. Then, each is deprotected to get the final product.

### Preparations using Alcohol Intermediate Route A

### Preparation 1

### 2-Benzyloxy-N-isopropyl-acetamide

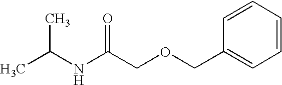

Add benzyloxyacetyl chloride (7.8 mL, 50 mmol) to a solution of isopropylamine (10.7 mL, 125 mmol) in dichloromethane (200 mL) at 0° C. After stirring at room temperature overnight, concentrate, partition between ethyl acetate and 1N HCl. Dry the organic phase (Na₂SO₄) and concentrate to give a white solid: 10.4 g. ¹H-NMR (CDCl₃) δ7.36 (m, 5H), 6.38 (bs, 1H), 4.56 (s, 2H), 4.11 (m, 1H), 3.95 (s, 2H), 1.73 (d, 6H).

### Preparation 2

### 2-Benzyloxy-N-isopropyl-thioacetamide

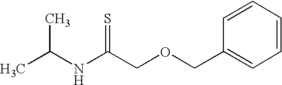

Add Lawesson's reagent (12.1 g, 30 mmol) to a suspension of 2-benzyloxy-N-isopropyl-acetamide (10.4 g, 50 mmol) in toluene (100 mL) and stir the mixture at reflux overnight. Evaporate to dryness, suspend the residue in Et₂O/hexanes, and filter. Concentrate the filtrate and purify by column chromatography (0-15%, EtOAc in hexanes) to give an oil: 10.5 g.

### Preparation 3

### 2-Benzyloxy-N-isopropyl-thioacetimidic acid methyl ester

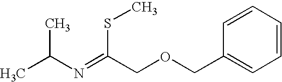

Add methyltriflate (10 g, 61 mmol) to a solution of 2-benzyloxy-N-isopropyl-thioacetamide (10 g, 45 mmol) in dichloromethane (200 mL) at 0° C. and stir the mixture at room temperature overnight. Evaporate the solvent to give a tan solid. ¹H-NMR (CDCl₃) δ 7.37 (m, 5H), 4.79 (s, 2H), 4.76 (s, 2H), 4.00 (m, 1H), 2.80 (s, 3H), 1.42 (d, 6H).

### Preparation 4

### 5-benzyloxymethyl-4-isopropyl-2-(4-trifluoromethyl-phenyl)-2,4-dihydro-[1,2,4]triazol-3-one

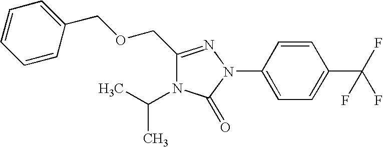

Stir a mixture of 2-benzyloxy-N-isopropyl-thioacetimidic acid methyl ester and 4-trifluoromethylphenyl hydrazine (7.9 g, 45 mmol) in pyridine (150 mL) at room temperature overnight. Evaporate the solvent and vacuum dry the residue to give an oil. Treat with carbonyl diimidazole (11 g, 67.5 mmol) in THF at reflux overnight. Dilute with ethyl acetate and washed with 1N HCl. Concentrate the organic phase and purify by column chromatography (0-20% EtOAc in hexanes) to give the desired product as an oil: 11 g. LC-MS: 392 (M+1).

### Preparation 5

### 5-Hydroxymethyl-4-isopropyl-2-(4-trifluoromethyl-phenyl)-2,4-dihydro-[1,2,4]triazol-3-one

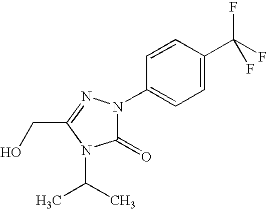

Subject a mixture of 5-benzyloxymethyl-4-isopropyl-2-(4-trifluoromethyl-phenyl)-2,4-dihydro-[1,2,4]triazol-3-one (8.5 g, 21.7 mmol), Pd(OH)₂/C (3.3 g) in ethanol (240 mL) to hydrogenolysis (H₂, 60 psi, 50° C., 18 hours). After filtration through a celite pad, concentrate the filtrate to give a white solid, 6.5 g. ¹H-NMR (DMSO-d₆) δ 8.13 (d, 2H), 7.82 (d, 2H), 5.86 (bs, 1H), 4.48 (s, 2H), 4.40 (m, 1H), 1.47 (d, 6H).

### Preparations using Alcohol Intermediate Route B

### Preparation 6

### Benzyloxy-acetic acid hydrazide

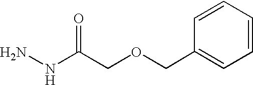

Heat a solution of benzyloxy-acetic acid methyl ester (45 g, 250 mmol) in hydrazinehydrate (25 mL) and ethanol (250 mL) to reflux for 4 hours. Cool the mixture to room temperature and concentrate to approximately 50 ml volume, then pour into a 1:1 mixture of water and diethyl ether (250 mL). Separate the mixture, and further extract the aqueous layer with ethyl acetate (2×200 mL). Wash the combined organic extracts with brine, dry over anhydrous sodium sulfate, filter, and concentrate to afford benzyloxy-acetic acid hydrazide, 35 g. ¹H NMR (CDCl₃) δ 7.26-7.36 (m, 5H), 4.56 (s, 2H), 4.06 (s, 2H).

### Preparation 7

### 5-Benzyloxymethyl-4-(2-fluoro-phenyl)-2,4-dihydro-[1,2,4]triazol-3-one

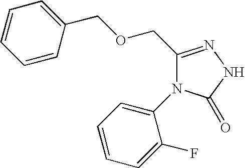

Add 2-Fluorophenyl isocyanate (2.49 ml, 22 2 mmol) to a solution of benzyloxy acetic acid hydrazide (4.0 g, 22.2 mmol) in THF (50 ml). Maintain the solution for 2 hours, then concentrate to afford the semicarbazide as a white solid, 7.04 g. ¹H NMR: (DMSO-d₆) δ 9.81 (s, 1H), 8.56 (s, 1H), 8.37 (s, 1H), 8.01 (m, 1H), 7.30-7.43 (m, 5H), 7.24 (m, 1H), 7.14 (m, 1H), 7.04 (m, 1H), 4.61 (s, 2H), 4.06 (s, 2H).

Add trimethylsilyltrifluoromethane sulfonate (5.97 ml, 33 0 mmol) to a solution of the product obtained above (3.5 g, 11.0 mmol) and triethylamine (7.67 ml, 55.0 mmol) in toluene (50 ml). Heat the mixture to reflux for 16 hours, then cool to 23° C. and pour the contents into saturated sodium bicarbonate (250 ml). Extract the mixture with diethyl ether (50 ml) and ethyl acetate (50 ml). Wash the combined organic extracts with saturated sodium chloride, dry over anhydrous sodium sulfate, filter, and concentrate. Purify the crude mixture by column chromatography (0 to 70% ethyl acetate/hexanes) to afford the 5-benzyloxymethyl-4-(2-fluoro-phenyl)-2,4-dihydro-[1,2,4]triazol-3-one (2.25 g). ¹H NMR (CDCl₃) δ 11.08 (s, 1H), 7.43-7.52 (m, 2H), 7.31 (m, 4H), 7.15 (m, 2H), 4.44 (s, 2H), 4.37 (s, 2H). ES-MS: 300 (M+1).

### Preparation 8

### 5-Benzyloxymethyl-4-(2-fluoro-phenyl)-2-(4-trifluoromethyl-phenyl)-2,4-dihydro-[1,2,4]triazol-3-one

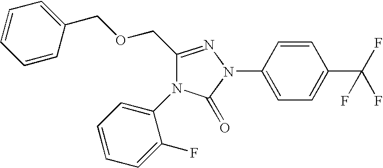

Bubble nitrogen gas through a mixture of 5-benzyloxymethyl-4-(2-fluoro-phenyl)-2,4-dihydro-[1,2,4]triazol-3-one (2.25 g, 7.52 mmol), 4-trifluoromethylphenyl iodide (1.24 ml, 8.63 mmol), and potassium carbonate (2.08 g, 15 0 mmol) in dioxane (15 ml) for 5 minutes. Add copper (I) iodide (0.072 g, 0.38 mmol) and trans-1,2-amino cyclohexane (0.086 g, 0.75 mmol) sequentially, then heat the reaction to reflux for 16 hours. Pour the contents into saturated sodium bicarbonate (50 ml). Extract the mixture with diethyl ether (2×25 ml) and ethyl acetate (2×25 ml). Wash the combined organic extracts with water (2×) and saturated sodium chloride, dry over anhydrous sodium sulfate, filter, and concentrate. Purify the crude mixture by column chromatography (0 to 20% ethyl acetate/hexanes) to afford the 5-benzyloxymethyl-4-(2-fluoro-phenyl)-2-(4-trifluoromethyl-phenyl)-2,4-dihydro-[1,2,4]triazol-3-one (2.68 g) as a white solid. ¹H ¹H-NMR (CDCl₃) δ 8.20 (d, 2H), 7.71 (d, 2H), 7.42-7.51 (m, 2H), 7.31 (m, 5H), 7.13 (m, 2H), 4.47 (s, 2H), 4.42 (s, 2H).

### Preparation 9

### 4-(2-Fluoro-phenyl)-5-hydroxymethyl-2-(4-trifluoromethyl-phenyl)-2,4-dihydro-[1,2,4]triazol-3-one

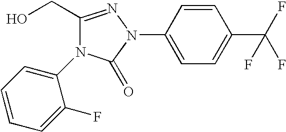

Obtain the titled compound from 5-benzyloxymethyl-4-(2-fluorophenyl)-2-(4-trifluoromethyl-phenyl)-2,4-dihydro-[1,2,4]triazol-3-one after hydrogenolysis using a similar protocol as described in Preparation 5. ¹H NMR (CDCl₃, 7.26) δ 8.21 (d, 2H), 7.73 (d, 2H), 7.56 (m, 2H), 7.37 (m, 2H), 4.58 (s, 2H), 2.09 (br s, 1H).

### Preparations of a Secondary Alcohol of Scheme 5

### Preparation 10

### 4-Isopropyl-5-oxo-1-(4-trifluoromethyl-phenyl)-4,5-dihydro-1H-[1,2,4]triazole-3-carbaldehyde

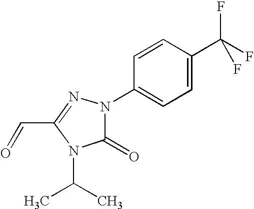

Stir a mixture of 5-hydroxymethyl-4-isopropyl-2-(4-trifluoromethyl-phenyl)-2,4-dihydro-[1,2,4]triazol-3-one (5.1 g, 17 mmol), pyridinium chlorochromate (18.3 g, 85 mmol), celite (18.3 g) and 4A molecular sieve (18.3 g) in dichloromethane (300 mL) at ambient temperature overnight. Filter through a celite pad and concentrate the filtrate to give a tan solid, 4 g. ¹H-NMR (CDCl₃) δ 9.64 (s, 1H), 8.21 (d, 2H), 7.73 (d, 2H), 5.05 (m, 1H), 1.56 (d, 6H).

### Preparation 11

### 5-(Hydroxy-pyridin-3-yl-methyl)-4-isopropyl-2-(4-trifluoromethyl-phenyl)-2,4-dihydro-[1,2,4]triazol-3-one

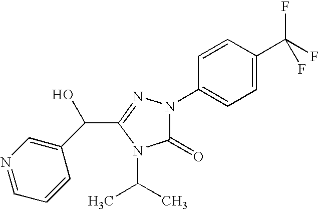

Add isopropylmagnesium chloride (1.34 mL, 2.67 mmol, 2.0M in THF) dropwise to an ambient temperature solution of 3-bromopyridine (262 μL, 2.67 mmol) in THF (3 mL). Stir the reaction at room temperature for 1 hour. Add triethylamine (372 μL, 2.67 mmol) followed by the dropwise addition of 4-Isopropyl-5-oxo-1-(4-trifluoromethyl-phenyl)-4,5-dihydro-1H-[1,2,4]triazole-3-carbaldehyde (800 mg, 2.67 mmol) in THF (3 mL) and stir the reaction at room temperature overnight. Quench the reaction with water and extract with Et₂O. Wash the combined organic layers with brine, dry (MgSO₄), filter, concentrate and chromatograph (5 to 40% EtOAC/Hex) to yield the title compound, 485 mg. ES-MS: 379 (M+1)

### Synthetic Method 1 (Scheme 1)

### Formation of the Ester Protected Intermediate Followed by Deprotection by Mitsunobu Reaction Followed by Base Hydrolysis

### Preparation 12

### 2-{4-[4-Isopropyl-5-oxo-1-(4-trifluoromethyl-phenyl)-4,5-dihydro-1H-[1,2,4]triazol-3-ylmethoxy]-2-methyl-phenoxy}-2-methyl-propionic acid ethyl ester

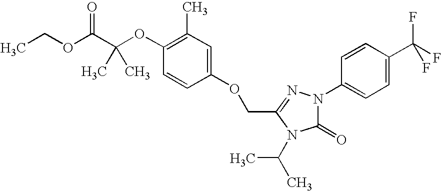

Add tri-n-butylphosphine (598 μl, 2.40 mmol), 2-(4-hydroxy-2-methyl-phenoxy)-2-methyl-propionic acid ethyl ester (374 mg, 1.57 mmol) to an ambient temperature solution of 5-hydroxymethyl-4-isopropyl-2-(4-trifluoromethyl-phenyl)-2,4-dihydro-[1,2,4]triazol-3-one (481 mg, 1.60 mmol) in toluene (10 ml) and cool to ?? ° C. Add 1,1′-(Azodicarbonyl)-dipiperidine (606 mg, 2.40 mmol) and warm the reaction to room temperature overnight. Dilute the mixture with hexanes (100 ml), filter, concentrate and chromatograph (120 g SiO₂, 0% to 15% EtOAc/hexanes) to yield the desired product, 601 mg. LC-MS: 522 (M+1).

### Example 1

### 2-{4-[4-Isopropyl-5-oxo-1-(4-trifluoromethyl-phenyl)-4,5-dihydro-1H-[1,2,4]triazol-3-ylmethoxy]-2-methyl-phenoxy}-2-methyl-propionic acid

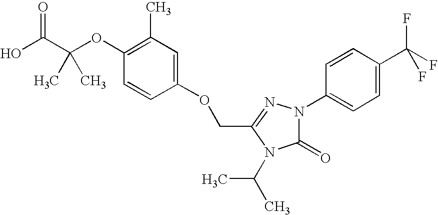

Add lithium hydroxide (1.69 ml, 3.36 mmol, 2.0 M in H₂O) to an ambient temperature solution of 2-{4-[4-Isopropyl-5-oxo-1-(4-trifluoromethyl-phenyl)-4,5-dihydro-1H-[1,2,4]triazol-3-ylmethoxy]-2-methyl-phenoxy}-2-methyl-propionic acid ethyl ester (588 mg, 1.12 mmol) in dioxane (3 ml) and heat to 50° C. overnight. Concentrate the mixture and partition the residue between Et₂O and 1 N HCl. Wash the organic phase with H₂O, dry (MgSO₄), filter and concentrate to yield the desired product, 539 mg. LC-MS: 494 (M+1).

### Synthetic Method 2 (Scheme 2)

### Formation of the Ester Protected Intermediate, N⁴-Deprotection Followed by Alkylation and Base Hydrolysis

### Preparation 13

### 2-Methyl-2-{2-methyl-4-[5-oxo-1-(4-trifluoromethyl-phenyl)-4,5-dihydro-1H-[1,2,4]triazol-3-ylmethoxy]-phenoxy}-propionic acid ethyl ester

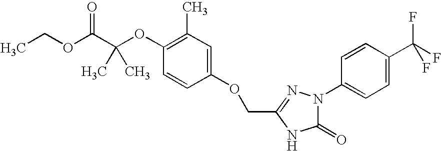

Add palladium tetrakis-triphenylphosphine (164 mg, 0,143 mmol) to a solution of 2-{4-[4-Allyl-5-oxo-1-(4-trifluoromethyl-phenyl)-4,5-dihydro-1H-[1,2,4]triazol-3-ylmethoxy]-2-methyl-phenoxy}-2-methyl-propionic acid ethyl ester (7.37 g, 14.19 mmol), triethylamine (4.9 mL, 35.47 mmol), and formic acid (1.1 mL, 28.38 mmol) in dioxane (47 mL). Degas the mixture several times by bubbling nitrogen and then heat to 85° C. for 18 h. Cool down to room temperature and filter through a pad of celite. Concentrate the filtrate and purify by column chromatography (10-30% EtOAc in hexanes) gives the titled product, 6.2 g. ¹H NMR (CDCl₃): 8.11 (d, 2H), 7,67 (d, 2H), 6.83 (s, 1H), 6.69 (m, 2H), 4,97 (s, 2H), 4,26 (c, 2H), 2,2 (s, 3H), 1,55 (s, 6H), 1,26 (t, 3H).

### Preparation 14

### 2-{4-[4-(2-Methoxy-ethyl)-5-oxo-1-(4-trifluoromethyl-phenyl)-4,5-dihydro-1H-[1,2,4]triazol-3-ylmethoxy]-2-methyl-phenoxy}-2-methyl-propionic acid ethyl ester

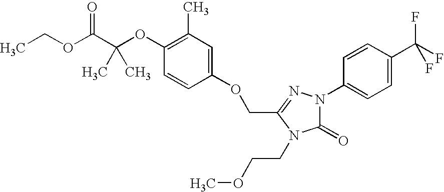

In a sealed tube, heat to reflux a mixture of 2-Methyl-2-{2-methyl-4-[5-oxo-1-(4-trifluoromethyl-phenyl)-4,5-dihydro-1H-[1,2,4]triazol-3-ylmethoxy]-phenoxy}-propionic acid ethyl ester (480 mg, 1 mmol), 1-Chloro-2-methoxy-ethane (146 μl, 1.6 mmol), diisopropyl ethyl amine (0.43 mL, 2.5 mmol), and sodium iodide (5 mg) in acetonitrile (2.5 mL) for 18 h. Cool the reaction to room temperature and evaporate the solvent under vacuum. Dissolve the residue in ethyl acetate. Wash the solution successively with a saturated solution of ammonium acetate, brine, and water. Dry the organic layer over magnesium sulfate, filter, and evaporate the solvents. Purify the crude by column chromatography in silica gel eluting with a gradient of ethyl acetate in hexanes (10-30%). to give the titled product, 529 mg. ¹H NMR (CDCl₃): 8.11 (d, 2H), 7.63 (d, 2H), 6.77 (d, 1H), 6.64 (m, 2H), 5.06(s, 2H), 4.22 (c, 2H), 4.01(t, 2H), 3.6 (t, 2H), 3.28 (s, 3H), 2.18 (s, 3H), 1.56 (s, 6H), 1.2 (t, 3H)

### Example 2

### 2-{4-[4-(2-Methoxy-ethyl)-5-oxo-1-(4-trifluoromethyl-phenyl)-4,5-dihydro-1H-[1,2,4]triazol-3-ylmethoxy]-2-methyl-phenoxy}-2-methyl-propionic acid

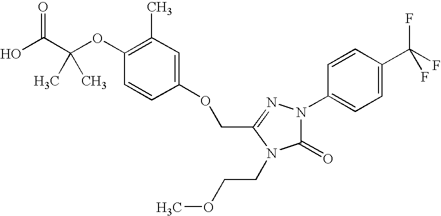

Dissolve 2-{4-[4-(2-Methoxy-ethyl)-5-oxo-1-(4-trifluoromethyl-phenyl)-4,5-dihydro-1H-[1,2,4]triazol-3-ylmethoxy]-2-methyl-phenoxy}-2-methyl-propionic acid ethyl ester (479 mg, 0.889 mmol) in 30 mL of a 1 to 1 mixture of THF and ethanol at room temperature. Add 2.2 mL of a 2N aqueous solution of potassium hydroxide and stir at room temperature for 18 h. Concentrate under vacuum and acidify to pH 4. Dilute with ethyl acetate and separate the phases. Extract the aqueous layer twice with ethyl acetate. Combine the organics and wash successively with brine and water, dry over magnesium sulfate, filter, and evaporate solvents to obtain the titled product, 417 mg. ES-MS: 510 (M+1).

In Table 1, the Examples are prepared essentially as described in Example 1 by preparing the Alcohol Intermediate Formula IIa by Route A (Scheme 3) and using Synthetic Method 1. Regarding substituents for structural Formula I set forth in the Brief Summary of the Invention, R¹ and R³ are as indicated and R² is hydrogen.

TABLE 1

| | | | MS | |
|---|---|---|---|---|
| Example | R¹ | R³ | (M + 1) | Name |
| 3 | —CH₃ | —CH₂CH₂CH₃ | 494 | 2-Methyl-2-{2-methyl-4-[5-oxo-4-propyl- |
| | | | | 1-(4-trifluoromethyl-phenyl)-4,5-dihydro- |
| | | | | 1H-[1,2,4]triazol-3-ylmethoxy]- |
| | | | | phenoxy}-propionic acid |
| 4 | —CH₃ | —CH₂— | 492 | 2-{4-[4-Allyl-5-oxo-1-(4-trifluoromethyl- |
| | | CH═CH₂ | | phenyl)-4,5-dihydro-1H-[1,2,4]triazol-3- |
| | | | | ylmethoxy]-2-methyl-phenoxy}-2-methyl- |
| | | | | propionic acid |
| 5 | —CH₃ | —CH₂CH₃ | 480 | 2- 4-[4-Ethyl-5-oxo-1-(4-trifluoromethyl- |
| | | | | phenyl)-4,5-dihydro-1H-[1,2,4]triazol-3- |
| | | | | ylmethoxy]-2-methyl-phenoxy}-2-methyl- |
| | | | | propionic acid |
| 6 | —CH₃ | —C(CH₃)₃ | 508 | 2- {4-[4-tert-Butyl-5-oxo-1-(4- |
| | | | | trifluoromethyl-phenyl)-4,5-dihydro-1H- |
| | | | | [1,2,4]triazol-3-ylmethoxy] -2-methyl- |
| | | | | phenoxy }-2-methyl-propionic acid |
| 7 | —CH₃ | —CH₃ | 466 | 2-Methyl-2-{2-methyl-4-[4-methyl-5-oxo- |
| | | | | 1-(4-trifluoromethyl-phenyl)-4,5-dihydro- |
| | | | | 1H-[1,2,4]triazol-3-ylmethoxy]- |
| | | | | phenoxy}-propionic acid |
| 8 | —CH₃ | 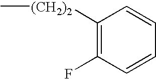 | 574 | 2-{4-[4-[2-(2-Fluoro-phenyl)-ethyl]-5- oxo-1-(4-trifluoromethyl-phenyl)-4,5- dihydro-1H-[1,2,4]triazol-3-ylmethoxy] 2-methyl-phenoxy}-2-methyl-propionic acid |
| 9 | —H | —CH(CH₃)₂ | 480 | 2-{4-[4-Isopropyl-5-oxo-1-(4- |
| | | | | trifluoromethyl-phenyl)-4,5-dihydro-1H- |
| | | | | [1,2,4]triazol-3-ylmethoxy]-phenoxy} -2- |
| | | | | methyl-propionic acid |
| 10 | —H | —CH₂CH₃ | 466 | 2-{4-[4-Ethyl-5-oxo-1-(4-trifluoromethyl- |
| | | | | phenyl)-4,5-dihydro-1H-[1,2,4]triazol-3- |
| | | | | ylmethoxy]-phenoxy}-2-methyl-propionic |
| | | | | acid |

In Table 2, the Examples are prepared essentially as described in Example 2 by preparing the Alcohol Intermediate Formula IIa by Route A (Scheme 3) and using the Synthetic Method 2. Regarding substituents for structural Formula I set forth in the Brief Summary of the Invention, R¹ and R³ are as indicated and R² is hydrogen.

TABLE 2

| | | | MS | |
|---|---|---|---|---|
| Example | R¹ | R³ | (M + 1) | Name |
| 11 | —CH₃ | —CH₂CH(CH₃)₂ | 508 | 2-{4-[4-Isobutyl-5-oxo-1-(4- |
| | | | | trifluoromethyl-phenyl)-4,5-dihydro-1H- |
| | | | | [1,2,4]triazol-3-ylmethoxy]-2-methyl- |
| | | | | phenoxy}-2-methyl-propionic acid |
| 12 | —CH₃ | 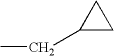 | 506 | 2-{4-[4-Cyclopropylmethyl-5-oxo-1-(4- trifluoromethyl-phenyl)-4,5-dihydro-1H- [1,2,4]triazol-3-ylmethoxy]-2-methyl- phenoxy}-2-methyl-propionic acid |

In Table 3, the Examples are prepared essentially as described in Example 1 by preparing the Alcohol Intermediate Formula IIa by Route B (Scheme 4) and using the Synthetic Method 1. Regarding substituents for structural Formula I set forth in the Brief Summary of the Invention, R¹ and R³ are as indicated and R² is hydrogen.

TABLE 3

| | | | MS | |
|---|---|---|---|---|
| Example | R¹ | R³ | (M + 1) | Name |
| 13 | —CH₃ | 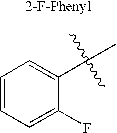 | 546 | 2- 4-[4-(2-Fluoro-phenyl)-5-oxo-1-(4- trifluoromethyl-phenyl)-4,5-dihydro-1H- [1,2,4]triazol-3-ylmethoxy] -2-methyl- phenoxy}-2-methyl-propionic acid |
| 14 | —H | 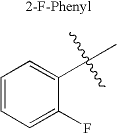 | 532 | 2-{4-[4-(2-Fluoro-phenyl)-5-oxo-1-(4- trifluoromethyl-phenyl)-4,5-dihydro-1H- [1,2,4]triazol-3 -ylmethoxy] -phenoxy}-2- methyl-propionic acid |
| 15 | —CH₃ | 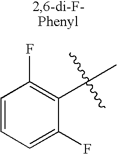 | 564 | 2-{4-[4-(2,6-Difluoro-phenyl)-5-oxo-1-(4- trifluoromethyl-phenyl)-4,5-dihydro-1H - [1,2,4]triazol-3-ylmethoxy]-2-methyl- phenoxy}-2-methyl-propionic acid |
| 16 | —H | 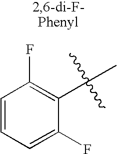 | 550 | 2-{4-[4-(2,6-Difluoro-phenyl)-5-oxo-1-(4- trifluoromethyl-phenyl)-4,5-dihydro-1H- [1,2,4]triazol-3-ylmethoxy]-phenoxy}-2- methyl-propionic acid |

In Table 4, the Examples are prepared essentially as described in Example 1 by preparing the Alcohol Intermediate Formula IIa by Route A (Scheme 3) and using the Synthetic Method 1. Regarding substituents for structural Formula I set forth in the Brief Summary of the Invention, R¹, R² and R³ are as indicated.

TABLE 4

| | | | | MS | |
|---|---|---|---|---|---|
| Example | R¹ | R² | R³ | (M + 1) | Name |
| 17 | —CH₃ | Phenyl | —CH₂CH₂CH₃ | 570 | 2-Methyl-2-(2-methyl-4-{ [5- |
| | | | | | oxo-4-propyl-1-(4- |
| | | | | | trifluoromethyl-phenyl)-4,5- |
| | | | | | dihydro-1H-[1,2,4]triazol-3- |
| | | | | | yl] -phenyl-methoxy} - |
| | | | | | phenoxy)-propionic acid |
| | | | | | (racemic) |
| 18 | —CH₃ | Phenyl | —CH₂CH₃ | 556 | 2-(4-{[4-Ethyl-5-oxo-1-(4- |
| | | | | | trifluoromethyl-phenyl)-4,5- |
| | | | | | dihydro-1H-[ l ,2,4]triazol-3 - |
| | | | | | yl]-phenyl-methoxy}-2- |
| | | | | | methyl-phenoxy)-2-methyl- |
| | | | | | propionic acid (racemic) |
| 21 | —CH₃ | Phenyl | —CH₃ | 542 | 2-Methyl-2-(2-methyl-4-{ [4- |
| | | | | | methyl-5-oxo-1-(4- |
| | | | | | trifluoromethyl-phenyl)-4,5- |
| | | | | | dihydro-1H-[ l,2,4]triazol-3 - |
| | | | | | yl]-phenyl-methoxy} - |
| | | | | | phenoxy)-propionic acid |
| | | | | | (racemic) |
| 25 | —CH₃ | 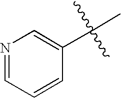 | —CH(CH₃)₂ | 571 | 2-(4-{4-Isopropyl-5-oxo-1-(4- trifluoromethyl-phenyl)-4,5- dihydro-1H-[1,2,4]triazol-3- yl]-pyridin-3-yl-methoxy}-2- methyl-phenoxy)-2-methyl- propionic acid (racemic) |
| 27 | —CH₃ | 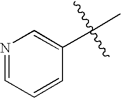 | —CH₂CH₂CH₃ | 571 | 2-Methyl-2-(2-methyl-4-{[5- oxo-4-propyl-1-(4- trifluoromethyl-phenyl)-4,5- dihydro-1H-[1,2,4]triazol-3- yl]-pyridin-3-yl-methoxy}- phenoxy)-propionic acid (racemic) |
| 28 | —CH₃ | 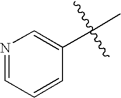 | —CH₂CH₃ | 557 | 2-(4-{[4-Ethyl-5-oxo-1-(4- trifluoromethyl-phenyl)-4,5- dihydro-1H-[1,2,4]triazol-3- yl]-pyridin-3-yl-methoxy}-2- methyl-phenoxy)-2-methyl- propionic acid (racemic) |
| 31 | —CH₃ | 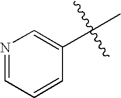 | —CH₂— CH═CH₂ | 569 | 2-(4-{[4-Allyl-5-oxo-1-(4- trifluoromethyl-phenyl)-4,5- dihydro-1H-[l,2,4]triazol-3- yl]-pyridin-3-yl-methoxy}-2- methyl-phenoxy)-2-methyl- propionic acid (racemic) |
| 35 | —H | Phenyl | —CH₃ | 528 | 2-Methyl-2-(4-{[4-methyl-5- |
| | | | | | oxo-1-(4-trifluoromethyl- |
| | | | | | phenyl)-4,5-dihydro-1H- |
| | | | | | [1,2,4]triazol-3-yl]-phenyl- |
| | | | | | methoxy}-phenoxy) -propionic |
| | | | | | acid (racemic) |
| 36 | —H | 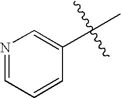 | —CH(CH₃)₂ | 557 | 2-(4-{[4-Isopropyl-5-oxo-1-(4- trifluoromethyl-phenyl)-4,5- dihydro-1H-[1,2,4]triazol-3- yl]-pyridin-3-yl-methoxy}- phenoxy)-2-methyl-propionic acid (racemic) |
| 37 | —H | —CH₃ | 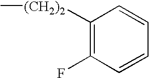 | 574 | 2-(4-{1-[4-[2-(2-Fluoro- phenyl)-ethyl]-5-oxo-1-(4- trifluoromethyl-phenyl)-4,5- dihydro-1H-[1,2,4]triazol-3- yl]-ethoxy}-phenoxy)-2- methyl-propionic acid (racemic) |

Table 4a: Isomers of Examples of Table 4. The following Examples are prepared by separating the racemic protected compound by chiral HPLC, collecting the protected isomers, and then deprotecting to get the Example.

TABLE 4a

| | Isomer of | | |
|---|---|---|---|
| | Example from | MS | |
| Example | Table 4 | (M + 1) | Name |
| 19 | Isomer-1 of | 556 | 2-(4-{[4-Ethyl-5-oxo-1-(4-trifluoromethyl-phenyl)- |
| | Example 18 | | 4,5-dihydro-1H-[1,2,4]triazol-3-yl]-phenyl- |
| | | | methoxy}-2-methyl-phenoxy)-2-methyl-propionic |
| | | | acid (isomer-1) |
| 20 | Isomer-2 of | 556 | 2-(4-{[4-Ethyl-5-oxo-1-(4-trifluoromethyl-phenyl)- |
| | Example 18 | | 4,5-dihydro-1H-[1,2,4]triazol-3-yl]-phenyl- |
| | | | methoxy}-2-methyl-phenoxy)-2-methyl-propionic |
| | | | acid (isomer-2) |
| 22 | Isomer-1 of | 542 | 2-Methyl-2-(2-methyl-4-{[4-methyl-5-oxo-1-(4- |
| | Example 21 | | trifluoromethyl-phenyl)-4,5-dihydro-1H- |
| | | | [1,2,4]triazol-3-yl]-phenyl-methoxy}-phenoxy)- |
| | | | propionic acid (isomer-1) |
| 26 | Isomer-2 of | 557 | 2-(4-{[4-Ethyl-5-oxo-1-(4-trifluoromethyl-phenyl)- |
| | Example 28 | | 4,5-dihydro-1H-[1,2,4]triazol-3-yl]-pyridin-3-yl- |
| | | | methoxy}-2-methyl-phenoxy)-2-methyl-propionic |
| | | | acid (isomer-2) |
| 29 | Isomer-1 of | 571 | 2-Methyl-2-(2-methyl-4-{[5-oxo-4-propyl-1-(4- |
| | Example 27 | | trifluoromethyl-phenyl)-4,5-dihydro-1H- |
| | | | [1,2,4]triazol-3-yl]-pyridin-3-yl-methoxy}- |
| | | | phenoxy)-propionic acid (isomer-1) |
| 30 | Isomer-1 of | 557 | 2-(4-{[4-Ethyl-5-oxo-1-(4-trifluoromethyl-phenyl)- |
| | Example 28 | | 4,5-dihydro-1H-[1,2,4]triazol-3-yl]-pyridin-3-yl- |
| | | | methoxy}-2-methyl-phenoxy)-2-methyl-propionic |
| | | | acid (isomer-1) |
| 32 | Isomer-1 of | 557 | 2-(4-{[4-Isopropyl-5-oxo-1-(4-trifluoromethyl- |
| | Example 36 | | phenyl)-4,5-dihydro-1H-[1,2,4]triazol-3-yl]- |
| | | | pyridin-3-yl-methoxy}-phenoxy)-2-methyl- |
| | | | propionic acid (Isomer-1) |
| 33 | Isomer-2 of | 571 | 2-Methyl-2-(2-methyl-4-{[5-oxo-4-propyl-1-(4- |
| | Example 27 | | trifluoromethyl-phenyl)-4,5-dihydro-1H- |
| | | | [1,2,4]triazol-3-yl]-pyridin-3-yl-methoxy}- |
| | | | phenoxy)-propionic acid (Isomer-2) |
| 34 | Isomer-2 of | 557 | 2-(4-{[4-Isopropyl-5-oxo-1-(4-trifluoromethyl- |
| | Example 36 | | phenyl)-4,5-dihydro-1H-[1,2,4]triazol-3-yl]- |
| | | | pyridin-3-yl-methoxy}-phenoxy)-2-methyl- |
| | | | propionic acid (isomer-2) |

In Table 5, the Examples are prepared essentially as described in Example 1 by preparing the Alcohol Intermediate Formula IIa by Route B (Scheme 4) and using the Synthetic Method 1. Regarding substituents for structural Formula I set forth in the Brief Summary of the Invention, R¹ and R² are as indicated and R³ is 2,6-di-fluoro-phenyl.

TABLE 5

| | | | MS | |
|---|---|---|---|---|
| Example | R¹ | R² | (M + 1) | Name |
| 38 | —H | —CH₂CH₂CH₃ | 592 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]- |
| | | | | butoxy}-phenoxy)-2-methyl-propionic |
| | | | | acid (racemic) |
| 42 | —CH₃ | —CH₂CH₂CH₃ | 606 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]- |
| | | | | butoxy}-2-methyl-phenoxy)-2-methyl- |
| | | | | propionic acid (racemic) |
| 45 | —CH₃ | —(CH₂)₃CH₃ | 620 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]- |
| | | | | pentyloxy}-2-methyl-phenoxy)-2- |
| | | | | methyl-propionic acid (racemic) |
| 46 | —H | —CH₃ | 564 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]- |
| | | | | ethoxy}-phenoxy)-2-methyl-propionic |
| | | | | acid (racemic) |
| 49 | —CH₃ | —CH₃ | 578 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]- |
| | | | | ethoxy}-2-methyl-phenoxy)-2-methyl- |
| | | | | propionic acid (racemic) |
| 51 | —H | —CH₂CH₂CF₃ | 646 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]-4,4,4- |
| | | | | trifluoro-butoxy}-phenoxy)-2-methyl- |
| | | | | propionic acid (racemic) |
| 54 | —CH₃ | —CH₂CH₂CF₃ | 660 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]-4,4,4- |
| | | | | trifluoro-butoxy}-2-methyl-phenoxy)- |
| | | | | 2-methyl-propionic acid (racemic) |
| 57 | —H | —(CH₂)₃CF₃ | 660 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]-5,5,5- |
| | | | | trifluoro-pentyloxy}-phenoxy)-2- |
| | | | | methyl-propionic acid (racemic) |
| 58 | —CH₃ | —(CH₂)₃CF₃ | 674 | 2-(4-{1-[4-(2,6-Difluoro-phenyl)-5- |
| | | | | oxo-1-(4-trifluoromethyl-phenyl)-4,5- |
| | | | | dihydro-1H-[1,2,4]triazol-3-yl]-5,5,5- |
| | | | | trifluoro-pentyloxy}-2-methyl- |
| | | | | phenoxy)-2-methyl-propionic acid |
| | | | | (racemic) |

Table 5a: Isomers of Examples of Table 5. The following Examples are prepared by separating the racemic protected compound by chiral HPLC, collecting the protected isomers, and then deprotecting to get the example.

TABLE 5a

| | Isomer of | | |
|---|---|---|---|
| | Example from | MS | |
| Example | Table 5 | (M + 1) | Name |
| 39 | Isomer-1 of | 592 | 2-(4-{1-[4-(2,6-Difluoro-phenyl)-5-oxo-1-(4- |
| | Example 38 | | trifluoromethyl-phenyl)-4,5-dihydro-1H- |
| | | | [1,2,4]triazol-3-yl]-butoxy}-phenoxy)-2- |
| | | | methyl-propionic acid (isomer-1) |
| 40 | Isomer-2 of | 592 | 2-(4-{1-[4-(2,6-Difluoro-phenyl)-5-oxo-1-(4- |
| | Example 38 | | trifluoromethyl-phenyl)-4,5-dihydro-1H- |
| | | | [1,2,4]triazol-3-yl]-butoxy}-phenoxy)-2- |
| | | | methyl-propionic acid (isomer-2) |
| 41 | Isomer-1 of | 606 | 2-(4-{1-[4-(2,6-Difluoro-phenyl)-5-oxo-1-(4- |
| | Example 42 | | trifluoromethyl-phenyl)-4,5-dihydro-1H- |
| | | | [1,2,4]triazol-3-yl]-butoxy}-2-methyl- |
| | | | phenoxy)-2-methyl-propionic acid (isomer-1) |
| 43 | Isomer-2 of | 606 | 2-(4-{1-[4-(2,6-Difluoro-phenyl)-5-oxo-1-(4- |
| | Example 42 | | trifluoromethyl-phenyl)-4,5-dihydro-1H- |
| | | | [1,2,4]triazol-3-yl]-butoxy}-2-methyl- |
| | | | phenoxy)-2-methyl-propionic acid (isomer-2) |
| 47 | Isomer-1 of | 564 | 2-(4-{1-[4-(2,6-Difluoro-phenyl)-5-oxo-1-(4- |
| | Example 46 | | trifluoromethyl-phenyl)-4,5-dihydro-1H- |
| | | | [1,2,4]triazol-3-yl]-ethoxy}-phenoxy)-2- |
| | | | methyl-propionic acid (isomer-1) |
| 48 | Isomer-2 of | 564 | 2-(4-{1-[4-(2,6-Difluoro-phenyl)-5-oxo-1-(4- |
| | Example 46 | | trifluoromethyl-phenyl)-4,5-dihydro-1H- |
| | | | [1,2,4]triazol-3-yl]-ethoxy}-phenoxy)-2- |
| | | | methyl-propionic acid (isomer-2) |
| 50 | Isomer-2 of | 578 | 2-(4-{1-[4-(2,6-Difluoro-phenyl)-5-oxo-1-(4- |
| | Example 49 | | trifluoromethyl-phenyl)-4,5-dihydro-1H- |
| | | | [1,2,4]triazol-3-yl]-ethoxy}-2-methyl- |
| | | | phenoxy)-2-methyl-propionic acid (isomer-2) |
| 52 | Isomer-1 of | 646 | 2-(4-{1-[4-(2,6-Difluoro-phenyl)-5-oxo-1-(4- |
| | Example 51 | | trifluoromethyl-phenyl)-4,5-dihydro-1H- |
| | | | [1,2,4]triazol-3-yl]-4,4,4-trifluoro-butoxy}- |
| | | | phenoxy)-2-methyl-propionic acid (isomer-1) |
| 53 | Isomer-2 of | 646 | 2-(4-{1-[4-(2,6-Difluoro-phenyl)-5-oxo-1-(4- |
| | Example 51 | | trifluoromethyl-phenyl)-4,5-dihydro-1H- |
| | | | [1,2,4]triazol-3-yl]-4,4,4-trifluoro-butoxy}- |
| | | | phenoxy)-2-methyl-propionic acid (isomer-2) |
| 55 | Isomer-1 of | 660 | 2-(4-{1-[4-(2,6-Difluoro-phenyl)-5-oxo-1-(4- |
| | Example 54 | | trifluoromethyl-phenyl)-4,5-dihydro-1H- |
| | | | [1,2,4]triazol-3-yl]-4,4,4-trifluoro-butoxy}-2- |
| | | | methyl-phenoxy)-2-methyl-propionic acid |
| | | | (isomer-1) |
| 56 | Isomer-2 of | 660 | 2-(4-{1-[4-(2,6-Difluoro-phenyl)-5-oxo-1-(4- |
| | Example 54 | | trifluoromethyl-phenyl)-4,5-dihydro-1H- |
| | | | [1,2,4]triazol-3-yl]-4,4,4-trifluoro-butoxy}-2- |
| | | | methyl-phenoxy)-2-methyl-propionic acid |
| | | | (Isomer-2) |
| 59 | Isomer-1 of | 674 | 2-(4-{1-[4-(2,6-Difluoro-phenyl)-5-oxo-1-(4- |
| | Example 58 | | trifluoromethyl-phenyl)-4,5-dihydro-1H- |
| | | | [1,2,4]triazol-3-yl]-5,5,5-trifluoro-pentyloxy}- |
| | | | 2-methyl-phenoxy)-2-methyl-propionic acid |
| | | | (isomer-1) |
| 60 | Isomer-2 of | 674 | 2-(4-{1-[4-(2,6-Difluoro-phenyl)-5-oxo-1-(4- |
| | Example 58 | | trifluoromethyl-phenyl)-4,5-dihydro-1H- |
| | | | [1,2,4]triazol-3-yl]-5,5,5-trifluoro-pentyloxy}- |
| | | | 2-methyl-phenoxy)-2-methyl-propionic acid |
| | | | (isomer-2) |

In Table 6, the Examples are prepared essentially as described in Example 1 by preparing the Alcohol Intermediate Formula IIa by Route B (Scheme 4) and using the Synthetic Method 1. Regarding substituents for structural Formula I set forth in the Brief Summary of the Invention, R¹ and R² are as indicated and R³ is 2-fluoro-phenyl.

TABLE 6

| | | | MS | |
|---|---|---|---|---|
| Example | R¹ | R² | (M + 1) | Name |
| 61 | —H | —CH₂CH₂CH₃ | 574 | 2-(4-{1-[4-(2-Fluoro-phenyl)-5-oxo-1- |
| | | | | (4-trifluoromethyl-phenyl)-4,5- |
| | | | | dihydro-1H-[1,2,4]triazol-3-yl]- |
| | | | | butoxy}-phenoxy)-2-methyl-propionic |
| | | | | acid (racemic) |
| 64 | —CH₃ | —CH₂CH₂CH₃ | 588 | 2-(4-{1-[4-(2-Fluoro-phenyl)-5-oxo-1- |
| | | | | (4-trifluoromethyl-phenyl)-4,5- |
| | | | | dihydro-1H-[1,2,4]triazol-3-yl]- |
| | | | | butoxy}-2-methyl-phenoxy)-2-methyl- |
| | | | | propionic acid (racemic) |
| 66 | —H | —(CH₂)₃CH₃ | 588 | 2-(4-{1-[4-(2-Fluoro-phenyl)-5-oxo-1- |
| | | | | (4-trifluoromethyl-phenyl)-4,5- |
| | | | | dihydro-1H-[1,2,4]triazol-3-yl]- |
| | | | | pentyloxy}-phenoxy)-2-methyl- |
| | | | | propionic acid (racemic) |
| 69 | —CH₃ | —(CH₂)₃CH₃ | 602 | 2-(4-{1-[4-(2-Fluoro-phenyl)-5-oxo-1- |
| | | | | (4-trifluoromethyl-phenyl)-4,5- |
| | | | | dihydro-1H-[1,2,4]triazol-3-yl]- |
| | | | | pentyloxy}-2-methyl-phenoxy)-2- |
| | | | | methyl-propionic acid (racemic) |
| 72 | —H | —CH₃ | 546 | 2-(4-{1-[4-(2-Fluoro-phenyl)-5-oxo-1- |
| | | | | (4-trifluoromethyl-phenyl)-4,5- |
| | | | | dihydro-1H-[1,2,4]triazol-3-yl]- |
| | | | | ethoxy}-phenoxy)-2-methyl-propionic |
| | | | | acid (racemic) |
| 74 | —H | —CH₂CH₃ | 560 | 2-(4-{1-[4-(2-Fluoro-phenyl)-5-oxo-1- |
| | | | | (4-trifluoromethyl-phenyl)-4,5- |
| | | | | dihydro-1H-[1,2,4]triazol-3-yl]- |
| | | | | propoxy}-phenoxy)-2-methyl- |
| | | | | propionic acid (racemic) |
| 77 | —H | —CH₂CH₂CF₃ | 628 | 2-Methyl-2-(4-{4,4,4-trifluoro-1-[4- |
| | | | | (2-fluoro-phenyl)-5-oxo-1-(4- |
| | | | | trifluoromethyl-phenyl)-4,5-dihydro- |
| | | | | 1H-[1,2,4]triazol-3-yl]-butoxy}- |
| | | | | phenoxy)-propionic acid (racemic) |
| 80 | —CH₃ | —CH₂CH₂CF₃ | 642 | 2-Methyl-2-(2-methyl-4-(4,4,4- |
| | | | | trifluoro-1-[4-(2-fluoro-phenyl)-5- |
| | | | | oxo-1-(4-trifluoromethyl-phenyl)-4,5- |
| | | | | dihydro-1H-[1,2,4]triazol-3-yl]- |
| | | | | butoxy}-phenoxy)-propionic acid |
| | | | | (racemic) |
| 82 | —H | —(CH₂)₃CF₃ | 642 | 2-Methyl-2-(4-{5,5,5-trifluoro-1-[4- |
| | | | | (2-fluoro-phenyl)-5-oxo-1-(4- |
| | | | | trifluoromethyl-phenyl)-4,5-dihydro- |
| | | | | 1H-[1,2,4]triazol-3-yl]-pentyloxy}- |
| | | | | phenoxy)-propionic acid (racemic) |
| 85 | —CH₃ | —(CH₂)₃CF₃ | 656 | 2-Methyl-2-(2-methyl-4-(5,5,5- |
| | | | | trifluoro-1-[4-(2-fluoro-phenyl)-5- |
| | | | | oxo-1-(4-trifluoromethyl-phenyl)-4,5- |
| | | | | dihydro-1H-[1,2,4]triazol-3-yl]- |
| | | | | pentyloxy}-phenoxy)-propionic acid |
| | | | | (racemic)) |

Table 6a: Isomers of Examples of Table 6. The following Examples are prepared by separating the racemic protected compound by chiral HPLC, collecting the protected isomers, and then deprotecting to get the example.

TABLE 6a

| | Isomer of | | |
|---|---|---|---|
| | Example from | MS | |
| Example | Table 6 | (M + 1) | Name |
| 62 | Isomer-1 of | 574 | 2-(4-{1-[4-(2-Fluoro-phenyl)-5-oxo-1-(4- |
| | Example 61 | | trifluoromethyl-phenyl)-4,5-dihydro-1H- |
| | | | [1,2,4]triazol-3-yl]-butoxy}-phenoxy)-2- |
| | | | methyl-propionic acid (isomer-1) |
| 63 | Isomer-2 of | 574 | 2-(4-{1-[4-(2-Fluoro-phenyl)-5-oxo-1-(4- |
| | Example 61 | | trifluoromethyl-phenyl)-4,5-dihydro-1H- |
| | | | [1,2,4]triazol-3-yl]-butoxy}-phenoxy)-2- |
| | | | methyl-propionic acid (isomer-2) |
| 65 | Isomer-1 of | 588 | 2-(4-{1-[4-(2-Fluoro-phenyl)-5-oxo-1-(4- |
| | Example 64 | | trifluoromethyl-phenyl)-4,5-dihydro-1H- |
| | | | [1,2,4]triazol-3-yl]-butoxy}-2-methyl- |
| | | | phenoxy)-2-methyl-propionic acid (isomer-1) |
| 67 | Isomer-1 of | 588 | 2-(4-{1-[4-(2-Fluoro-phenyl)-5-oxo-1-(4- |
| | Example 66 | | trifluoromethyl-phenyl)-4,5-dihydro-1H- |
| | | | [1,2,4]triazol-3-yl]-pentyloxy}-phenoxy)-2- |
| | | | methyl-propionic acid (isomer-1) |
| 68 | Isomer-2 of | 588 | 2-(4-{1-[4-(2-Fluoro-phenyl)-5-oxo-1-(4- |
| | Example 66 | | trifluoromethyl-phenyl)-4,5-dihydro-1H- |
| | | | [1,2,4]triazol-3-yl]-pentyloxy}-phenoxy)-2- |
| | | | methyl-propionic acid (isomer-2) |
| 70 | Isomer-1 of | 602 | 2-(4-{1-[4-(2-Fluoro-phenyl)-5-oxo-1-(4- |
| | Example 69 | | trifluoromethyl-phenyl)-4,5-dihydro-1H- |
| | | | [1,2,4]triazol-3-yl]-pentyloxy}-2-methyl- |
| | | | phenoxy)-2-methyl-propionic acid (isomer-1) |
| 71 | Isomer-2 of | 602 | 2-(4-{1-[4-(2-Fluoro-phenyl)-5-oxo-1-(4- |
| | Example 69 | | trifluoromethyl-phenyl)-4,5-dihydro-1H- |
| | | | [1,2,4]triazol-3-yl]-pentyloxy}-2-methyl- |
| | | | phenoxy)-2-methyl-propionic acid (isomer-2) |
| 73 | Isomer-2 of | 546 | 2-(4-{1-[4-(2-Fluoro-phenyl)-5-oxo-1-(4- |
| | Example 72 | | trifluoromethyl-phenyl)-4,5-dihydro-1H- |
| | | | [1,2,4]triazol-3-yl]-ethoxy}-phenoxy)-2- |
| | | | methyl-propionic acid (isomer-2) |
| 75 | Isomer-1 of | 560 | 2-(4-{1-[4-(2-Fluoro-phenyl)-5-oxo-1-(4- |
| | Example 74 | | trifluoromethyl-phenyl)-4,5-dihydro-1H- |
| | | | [1,2,4]triazol-3-yl]-propoxy}-phenoxy)-2- |
| | | | methyl-propionic acid (isomer-1) |
| 76 | Isomer-2 of | 560 | 2-(4-{1-[4-(2-Fluoro-phenyl)-5-oxo-1-(4- |
| | Example 74 | | trifluoromethyl-phenyl)-4,5-dihydro-1H- |
| | | | [1,2,4]triazol-3-yl]-propoxy}-phenoxy)-2- |
| | | | methyl-propionic acid (isomer-2) |
| 78 | Isomer-1 of | 628 | 2-Methyl-2-(4-{4,4,4-trifluoro-1-[4-(2-fluoro- |
| | Example 77 | | phenyl)-5-oxo-1-(4-trifluoromethyl-phenyl)- |
| | | | 4,5-dihydro-1H-[1,2,4]triazol-3-yl]-butoxy}- |
| | | | phenoxy)-propionic acid (isomer-1) |
| 79 | Isomer-2 of | 628 | 2-Methyl-2-(4-{4,4,4-trifluoro-1-[4-(2-fluoro- |
| | Example 77 | | phenyl)-5-oxo-1-(4-trifluoromethyl-phenyl)- |
| | | | 4,5-dihydro-1H-[1,2,4]triazol-3-yl]-butoxy}- |
| | | | phenoxy)-propionic acid (isomer-2) |
| 81 | Isomer-1 of | 642 | 2-Methyl-2-(2-methyl-4-{4,4,4-trifluoro-1-[4- |
| | Example 80 | | (2-fluoro-phenyl)-5-oxo-1-(4-trifluoromethyl- |
| | | | phenyl)-4,5-dihydro-1H-[1,2,4]triazol-3-yl]- |
| | | | butoxy}-phenoxy)-propionic acid (isomer-1) |
| 83 | Isomer-1 of | 642 | 2-Methyl-2-(4-{5,5,5-trifluoro-1-[4-(2-fluoro- |
| | Example 82 | | phenyl)-5-oxo-1-(4-trifluoromethyl-phenyl)- |
| | | | 4,5-dihydro-1H-[1,2,4]triazol-3-yl]-pentyloxy}- |
| | | | phenoxy)-propionic acid (isomer-1) |
| 84 | Isomer-2 of | 642 | 2-Methyl-2-(4-{5,5,5-trifluoro-1-[4-(2-fluoro- |
| | Example 82 | | phenyl)-5-oxo-1-(4-trifluoromethyl-phenyl)- |
| | | | 4,5-dihydro-1H-[1,2,4]triazol-3-yl]-pentyloxy}- |
| | | | phenoxy)-propionic acid (isomer-2) |
| 86 | Isomer-1 of | 656 | 2-Methyl-2-(2-methyl-4-{5,5,5-trifluoro-1-[4- |
| | Example 85 | | (2-fluoro-phenyl)-5-oxo-1-(4-trifluoromethyl- |
| | | | phenyl)-4,5-dihydro-1H-[1,2,4]triazol-3-yl]- |
| | | | pentyloxy}-phenoxy)-propionic acid (isomer-1) |
| 87 | Isomer-2 of | 656 | 2-Methyl-2-(2-methyl-4-{5,5,5-trifluoro-1-[4- |
| | Example 85 | | (2-fluoro-phenyl)-5-oxo-1-(4-trifluoromethyl- |
| | | | phenyl)-4,5-dihydro-1H-[1,2,4]triazol-3-yl]- |
| | | | pentyloxy}-phenoxy)-propionic acid (isomer-2) |

### Biological Assays

### Binding and Cotransfection Studies

The in vitro potency of compounds in modulating PPARα receptors are determined by the procedures detailed below. DNA-dependent binding (ABCD binding) is carried out using SPA technology with PPAR receptors. Tritium-labeled PPARCX agonists are used as radioligands for generating displacement curves and IC₅₀ values with compounds of the invention. Cotransfection assays are carried out in CV-1 cells. The reporter plasmid contains an acylCoA oxidase (AOX) PPRE and TK promoter upstream of the luciferase reporter cDNA. Appropriate PPARs are constitutively expressed using plasmids containing the CMV promoter. For PPARα, interference by endogenous PPARγ in CV-1 cells is an issue. In order to eliminate such interference, a GAL4 chimeric system is used in which the DNA binding domain of the transfected PPAR is replaced by that of GAL4, and the GAL4 response element is utilized in place of the AOX PPRE. Cotransfection efficacy is determined relative to PPARα agonist reference molecules. Efficacies are determined by computer fit to a concentration-response curve, or in some cases at a single high concentration of agonist (10 μM).

These studies are carried out to evaluate the ability of compounds of the invention to bind to and/or activate various nuclear transcription factors, particularly huPPARα (“hu” indicates “human”). These studies provide in vitro data concerning efficacy and selectivity of compounds of the invention. Furthermore, binding and cotransfection data for compounds of the invention are compared with corresponding data for marketed compounds that act on huPPARα.

### PXR Assay: GAL4PXR/GAL4 Response Element Reporter Assays in HuH7 Cells:

Human liver HuH7 cells are co-transfected using Fugene. The reporter plasmid, containing five Gal4 binding site and major late promoter of adenovirus upstream of the luciferase reporter cDNA, is transfected with a plasmid constitutively expressing a hybrid receptor consistent of GAL4 DNA binding domain and human SXR ligand binding domain using viral SV40 early promoter. Cells are transfected with 10 μg of total DNA/10⁶ cells in T225 cm² flasks in DMEM:F12 (3:1) media with 10% charcoal-stripped Fetal Bovine Serum (FBS). After an overnight incubation, transfected cells are trypsinized, plated in 96 well dishes in DMEM:F12 (3:1) media with 10% charcoal-stripped FBS, incubated for 4 h and then exposed to 0.8 nM to 50 μM of test compounds in half log dilutions. After 24 h of incubations with compounds, cells are lysed and luciferase activity is determined Data is fit to a 4 parameter-fit logistics to determine EC₅₀ values. The % efficacy is determined versus maximum stimulation obtained with rifampicin.

All of the examples disclosed herein demonstrate activity in the binding assay with an EC₅₀ of less than 600 nM for PPARδ receptor and less than 3000 nM for the PPARα receptor. All of the examples disclosed herein demonstrate activity in the PXR assay with % efficacy (versus max stimulation with Rifampicin) of less than 70. Representative data for the example compounds in the binding assays are shown in Table 7 below.

TABLE 7

| | PPARδ | PPARα | PXR (% efficacy versus |
|---|---|---|---|
| | EC₅₀ | EC₅₀ | max stimulation with |
| Example | (nM) | (nM) | Rifampicin) |
| 1 | 63.4 | 308.9 | 32.4 |
| 2 | 296.9 | 256.1 | 52.8 |
| 13 | 14.0 | 19.5 | 57.8 |
| 57 | 46.4 | 872.1 | 13.5 |

### REFERENCES

Current Topics in Medicinal Chemistry. 3(14):1649-61 (2003).
Oliver, W. R. et al. Proc Natl Acad Sci 98:5306-5311 (2001).
Handschin, Pharmacology Reviews, vol. 55:4 p. 665 citing Jones et al. The pregnane X receptor Mol. Endocrinol. 14:27-39 (2000).
Barish, G. D. et al., The Journal of Clinical Investigation 116(3):590-597 (2006).

We claim:

1. A compound of the formula: 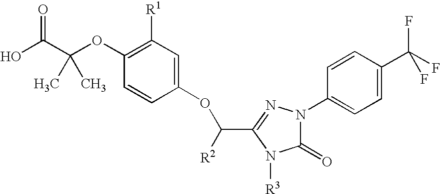
wherein:
R¹ is —H or —C₁-C₃ alkyl;
R² is selected from the group consisting of —H, —C₁-C₄ alkyl, —C₁-C₃ alkyl-CF₃, phenyl, and pyridinyl; and
R³ is selected from the group consisting of —H, —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines;
provided that when R¹ and R² are each H, then R³ is selected from the group consisting of —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines; or
stereoisomers and pharmaceutically acceptable salts thereof.

2. A compound of claim 1 wherein R¹ is methyl.

3. A compound of claim 2 wherein R² is H.

4. A compound as claimed by claim 3 wherein R³ is —C₁-C₄ alkyl.

5. A compound as claimed by claim 4 wherein R³ is C₃ alkyl.

6. A compound of claim 1 wherein
R¹ is —H or —CH₃;
R² is selected from the group consisting of —H, —C₁-C₄ alkyl, and —C₁-C₃ alkyl-CF₃; and
R³ is selected from the group consisting of —C₁-C₄ alkyl, —CH₂-cyclopropyl, —CH₂—C═CH₂, and phenyl substituted with 1 or 2 fluorines; or
stereoisomers and pharmaceutically acceptable salts thereof.

7. A compound of claim 1 that is 2-{4-[4-Isopropyl-5-oxo-1-(4-trifluoromethyl-phenyl)-4,5-dihydro-1H-[1,2,4]triazol-3-ylmethoxy]-2-methyl-phenoxy}-2-methyl-propionic acid.

8. A pharmaceutical formulation comprising a pharmaceutically acceptable carrier and at least one compound as claimed by claim 1.

9. A method for treating cardiovascular disease in a mammal, comprising the step of administering to the mammal a therapeutically effective amount of a compound as claimed by claim 1.

10. A method for lowering triglycerides in a mammal, comprising the step of administering to the mammal a therapeutically effective amount of a compound as claimed by claim 1.

11. A method for lowering blood glucose levels in a mammal comprising the step of administering a therapeutically effective amount of a compound as claimed by claim 1.

12. (canceled)

13. (canceled)

14. An intermediate for preparing a compound of claim 1 wherein the intermediate is: 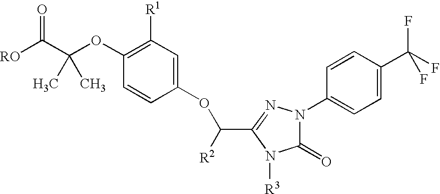
wherein:
R is —C₁-C₃ alkyl;
R¹ is —H or —C₁-C₃ alkyl;
R² is selected from the group consisting of —H, —C₁-C₄ alkyl, —C₁-C₃ alkyl-CF₃, phenyl, and pyridinyl; and
R³ is selected from the group consisting of —H, —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines;
provided that when R¹ and R² are each H, then R³ is selected from the group consisting of —C₁-C₄ alkyl, —C₁-C₃ alkyl-O—CH₃, —CH₂-cyclopropyl, —CH₂—C═CH₂, —CH₂CH₂-(2-F-phenyl), and phenyl substituted with from 1 to 2 fluorines, or
stereoisomers and pharmaceutically acceptable salts thereof.
